(19) United States
(12) Patent Application Publication
(10) Pub. No.: US 2016/0324584 A1
(43) Pub. Date: Nov. 10, 2016

(54) ULTRASOUND NAVIGATION/TISSUE CHARACTERIZATION COMBINATION

(71) Applicant: KONINKLIJKE PHILIPS N.V., Eindhoven (NL)

(72) Inventors: AMIR MOHAMMAD TAHMASEBI MARAGHOOSH, CAMBRIDGE, MA (US); AMEET KUMAR JAIN, CAMBRIDGE, MA (US); FRANCOIS GUY GERARD MARIE VIGNON, CAMBRIDGE, MA (US)

(21) Appl. No.: 15/109,330

(22) PCT Filed: Dec. 26, 2014
(86) PCT No.: PCT/IB2014/067337

§ 371 Date: Jun. 30, 2016

Related U.S. Application Data
(60) Provisional application No. 61/922,883, filed on Jan. 2, 2014.

(51) Int. Cl. A61B 34/20; A61B 8/00; A61B 90/00; A61B 8/08

(52) U.S. Cl. CPC A61B 34/20; A61B 8/0841; A61B 8/5223; A61B 8/463; A61B 90/37; A61B 2090/378; A61B 2034/2063; A61B 2034/2051; A61B 2034/2055

(57) ABSTRACT

A tool navigation system employing an ultrasound imager (21), a tool tracker (41), a tissue classifier (50) and an image navigator (60). In operation, ultrasound imager (21) generates an ultrasound image of an anatomical region from a scan of the anatomical region by an ultrasound probe (20). As an interventional tool (40) is navigated within the anatomical region, the tool tracker (41) tracks a position of the interventional tool (40) relative to the anatomical region, tissue classifier (50) characterizes the tissue of the anatomical region adjacent the interventional tool (40), and image navigator (60) displays a navigational guide relative to a display of the ultrasound image of the anatomical region. The navigational guide illustrates a position tracking of the interventional tool (40) for spatial guidance of the interventional tool (40) within the anatomical region and further illustrates a tissue characterization of the anatomical region for target guidance of the interventional tool (40) to a target location within the anatomical region.

The present invention generally relates to displaying a tracking of an interventional tool (e.g., a needle or catheter) within an ultrasound image of an anatomical region for facilitating a navigation of the interventional tool within the anatomical region. The present invention specifically relates to enhancing the tool tracking display by combining global information indicating a precise localization of the interventional tool within the ultrasound image of the anatomical region for spatial guidance of the interventional tool within the anatomical region, and local information indicating a characterization of tissue adjacent the interventional tool (e.g., tissue encircling the tool tip) for target guidance of the interventional tool to a target location within the anatomical region.

Tissue characterization is known as a medical procedure that assists in differentiating a structure and/or a function of a specific anatomical region of a body, human or animal. The structural/functional differentiation may be one between normality and abnormality, or may be concerned with changes over period of time associated with processes such as tumor growth or tumor response to radiation.

A number of techniques have been proposed for tissue characterization (e.g., MR spectroscopy, light/fluorescence spectroscopy, acoustic backscatter analysis, acoustic impedance-based, and electrical impedance-based tissue characterization). For example, a material's ability to conduct electrical current and to store electrical energy, also known as the material's impedance, differs between different materials. Biological tissues are no exception, and different tissues have different electrical impedance properties. Using the impedance of tissues, it has been shown that tumors differ from their surrounding healthy tissue.

More particularly, ultrasound-based tissue characterization is a well-studied problem. Nonetheless, ultrasound tissue characterization deep into an organ from pulse-echo data is challenging due to the fact that interactions between a biological tissue, which is an inhomogeneous medium, and an acoustic wave is very difficult to model. In particular, factors such as signal attenuation, which is frequency dependent, and beam diffraction, which makes the spatial and spectral beam characteristics depth dependent, affect the estimation of key parameters such as ultrasound backscatter. This has meant that ultrasound-based tissue characterization is not always strictly quantitative. Furthermore, most of the well-known tissue characterization techniques are not suitable for real-time procedures (e.g., different types of biopsies or minimal invasive surgeries) due to a complexity and a high price of running in real-time (e.g., MR spectroscopy) and/or due to a lack of localization information required to navigate the interventional tool to the target location within the anatomical region (e.g., light spectroscopy).

The present invention offers a combination of global information indicating a precise localization of an interventional tool on an ultrasound image for spatial guidance (e.g., tracking of a tip of the interventional tool within the ultrasound image) and of local information indicating a characterization of tissue adjacent the interventional tool for target guidance (e.g., identification and/or differentiation of tissue encircling a tip of the interventional tool). The combination of these two sources of information is expected to enhance the physician knowledge of the tissues the needle is going through to thereby improve surgical outcomes and reduce complications.

One form of the present invention is a tool navigation system employing an ultrasound probe (e.g., a 2D ultrasound probe), an ultrasound imager, an interventional tool (e.g., a needle or a catheter), a tool tracker, a tissue classifier and an image navigator. In operation, the ultrasound imager generates an ultrasound image of an anatomical region from a scan of the anatomical region by the ultrasound probe. As the interventional tool is navigated within the anatomical region, the tool tracker tracks a position of the interventional tool relative to the anatomical region (i.e., a location and/or an orientation of a tip of the interventional tool relative to the anatomical region), and the tissue classifier characterizes tissue adjacent the interventional tool (e.g., tissue encircling a tip of the interventional tool). The image navigator displays a navigational guide relative to a display of the ultrasound image of the anatomical region (e.g., a navigational overlay on a display of the ultrasound image of the anatomical region). The navigational guide simultaneously illustrates a position tracking of the interventional tool by the tool tracker for spatial guidance of the interventional tool within the anatomical region and a tissue characterization of the anatomical region by the tissue classifier for target guidance of the interventional tool to a target location within the anatomical region.

For tool tracking purposes, the tool navigation system can employ position sensor(s) operably connecting the interventional tool to the tool tracker to facilitate the position tracking by the tool tracker for spatial guidance of the interventional tool within the anatomical region. Examples of the positions sensor(s) include, but are not limited to, acoustic sensors(s), ultrasound transducer(s), electromagnetic sensor(s), optical sensor(s) and/or optical fiber(s). In particular, acoustic tracking of the interventional tool takes advantage of the acoustic energy emitted by the ultrasound probe as a basis for tracking the interventional tool.

For tissue characterization purposes, the tool navigation system can employ tissue sensor(s) operably connecting the interventional tool to the tissue classifier to facilitate the tissue classifier in identifying and differentiating tissue adjacent the interventional tool for target guidance of the interventional tool to a target location within the anatomical region. Examples of the tissue sensor(s) include, but are not limited to, acoustic sensor(s), ultrasound transducer(s), PZT mircosensor(s) and/or fiber optic hydrophone(s). In particular, fiber optic sensing of the tissue takes advantage of optical spectroscopy techniques for identifying and differentiating tissue adjacent the interventional tool.

For various embodiments of the tool navigation system, one or more of the sensors can serve as a position sensor and/or a tissue sensor.

Furthermore, alternatively or concurrently to employing the tissue sensor(s), the tissue classifier can identify and differentiate tissue within an image of the anatomical region to thereby map the tissue characterization of the anatomical region for target guidance of the interventional tool to a target location within the anatomical region (e.g., a tissue characterization map of the ultrasound image of the anatomical region, of a photo-acoustic image of the anatomical region and/or of a registered pre-operative image of the anatomical region).

For the navigation guide, the tool navigation guide can employ one or more of various display techniques including, but not limited to, overlays, side-by-side, color coding, time series tablet and beamed to big monitor. In particular, the navigation guide can be a graphical icon of the interventional tool employed to illustrate the position tracking of the interventional tool by the tool tracker and/or the tissue characterization of the anatomical region by the tissue classifier.

The image navigator can modulate one or more feature(s) of the graphical icon responsive to any change to a tissue type of the tissue characterization of the anatomical region by the tissue classifier. Alternatively or concurrently, a tissue characterization map illustrating a plurality of tissue types can be overlain on the ultrasound image of the anatomical region. In the alternative, the graphical icon may only illustrate the position tracking of the interventional tool by the tool tracker and can be modulated as the graphical icon approaches the target location within the anatomical region as illustrated in the tissue characterization map.

Another form of the present invention is a tool navigation system employing an ultrasound imager, a tool tracker, a tissue classifier and an image navigator. In operation, the ultrasound imager generates an ultrasound image of an anatomical region from a scan of the anatomical region by an ultrasound probe. As an interventional tool is navigated within the anatomical region, the tool tracker tracks a position of the interventional tool relative to the anatomical region (i.e., a location and/or an orientation of a tip of the interventional tool relative to the anatomical region), and the tissue classifier characterizes tissue adjacent the interventional tool (e.g., tissue encircling a tip of the interventional tool). The image navigator displays a navigational guide relative to a display of the ultrasound image of the anatomical region (e.g., a navigational overlay on a display of the ultrasound image of the anatomical region). The navigational guide simultaneously illustrates a position tracking of the interventional tool by the tool tracker for spatial guidance of the interventional tool within the anatomical region and a tissue characterization of the anatomical region by the tissue classifier for target guidance of the interventional tool to a target location within the anatomical region.

For tool tracking purposes, the tool navigation system can employ position sensor(s) operably connecting the interventional tool to the tool tracker to facilitate the position tracking by the tool tracker for spatial guidance of the interventional tool within the anatomical region. Examples of the positions sensor(s) include, but are not limited to, acoustic sensors(s), ultrasound transducer(s), electromagnetic sensor(s), optical sensor(s) and/or optical fiber(s). In particular, acoustic tracking of the interventional tool takes advantage of the acoustic energy emitted by the ultrasound probe as a basis for tracking the interventional tool.

For tissue characterization purposes, the tool navigation system can employ tissue sensor(s) operably connecting the interventional tool to the tissue classifier to facilitate the tissue classifier in identifying and differentiating tissue adjacent the interventional tool for target guidance of the interventional tool to a target location within the anatomical region. Examples of the tissue sensor(s) include, but are not limited to, acoustic sensor(s), ultrasound transducer(s), PZT mircosensor(s) and/or fiber optic hydrophone(s). In particular, fiber optic sensing of the tissue takes advantage of optical spectroscopy techniques for identifying and differentiating tissue adjacent the interventional tool.

For various embodiments of the tool navigation system, one or more of the sensors can serve as a position sensor and/or a tissue sensor.

Furthermore, alternatively or concurrently to employing the tissue sensor(s), the tissue classifier can identify and differentiate tissue within an image of the anatomical region to thereby map the tissue characterization of the anatomical region for target guidance of the interventional tool to a target location within the anatomical region (e.g., a tissue characterization map of the ultrasound image of the anatomical region, of a photo-acoustic image of the anatomical region and/or of a registered pre-operative image of the anatomical region).

For the navigation guide, the tool navigation guide can employ one or more of various display techniques including, but not limited to, overlays, side-by-side, color coding, time series tablet and beamed to big monitor. In particular, the navigation guide can be a graphical icon of the interventional tool employed to illustrate the position tracking of the interventional tool by the tool tracker and/or the tissue characterization of the anatomical region by the tissue classifier.

The image navigator can modulate one or more feature(s) of the graphical icon responsive to any change to a tissue type of the tissue characterization of the anatomical region by the tissue classifier. Alternatively or concurrently, a tissue characterization map illustrating a plurality of tissue types can be overlain on the ultrasound image of the anatomical region. In the alternative, the graphical icon can only illustrate the position tracking of the interventional tool by the tool tracker and be modulated and/or otherwise provide a graphical indication as the graphical icon approaches the target location within the anatomical region as illustrated in the tissue characterization map.

Another form of the present invention is a tool navigation method which includes generating an ultrasound image of an anatomical region from a scan of the anatomical region. As an interventional tool (e.g., a needle or a catheter) is navigated within the anatomical region, the method further includes tracking a position of the interventional tool relative to the anatomical region, characterizing tissue of the anatomical region adjacent the interventional tool, and displaying a navigational guide relative to a display of the ultrasound image of the anatomical region. The navigational guide simultaneously illustrates a position tracking of the interventional tool for spatial guidance of the interventional tool within the anatomical region, and a tissue characterization of the anatomical region for target guidance of the interventional tool to a target location within the anatomical region.

The foregoing forms and other forms of the present invention as well as various features and advantages of the present invention will become further apparent from the following detailed description of various embodiments of the present invention read in conjunction with the accompanying drawings. The detailed description and drawings are merely illustrative of the present invention rather than limiting, the scope of the present invention being defined by the appended claims and equivalents thereof.

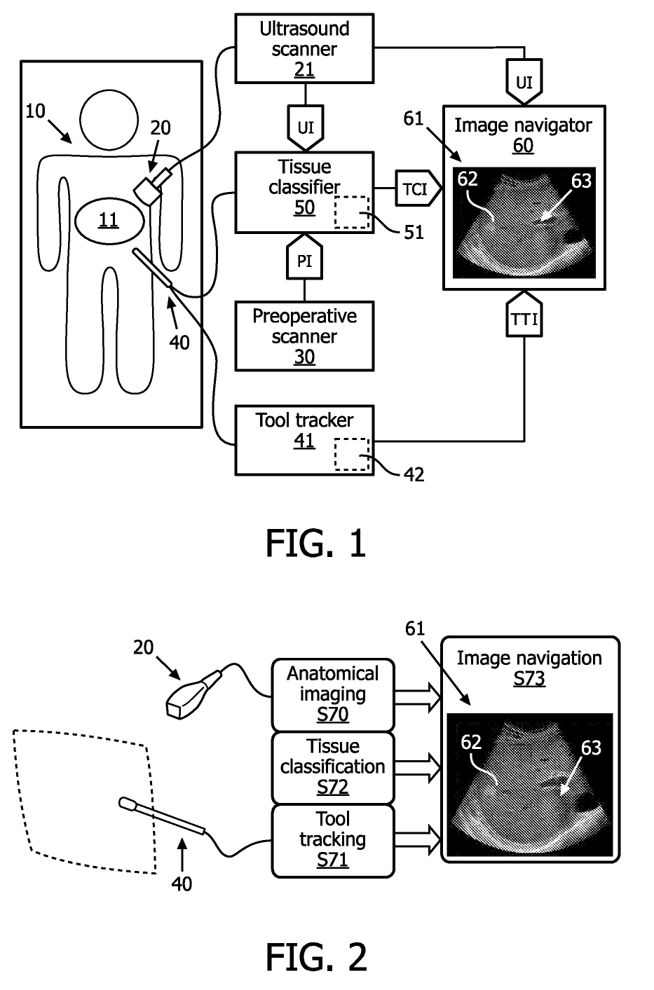

FIG. 1 illustrates an exemplary embodiment of tool navigation system in accordance with the present invention.

FIG. 2 illustrates an exemplary embodiment of a tool navigation method in accordance with the present invention.

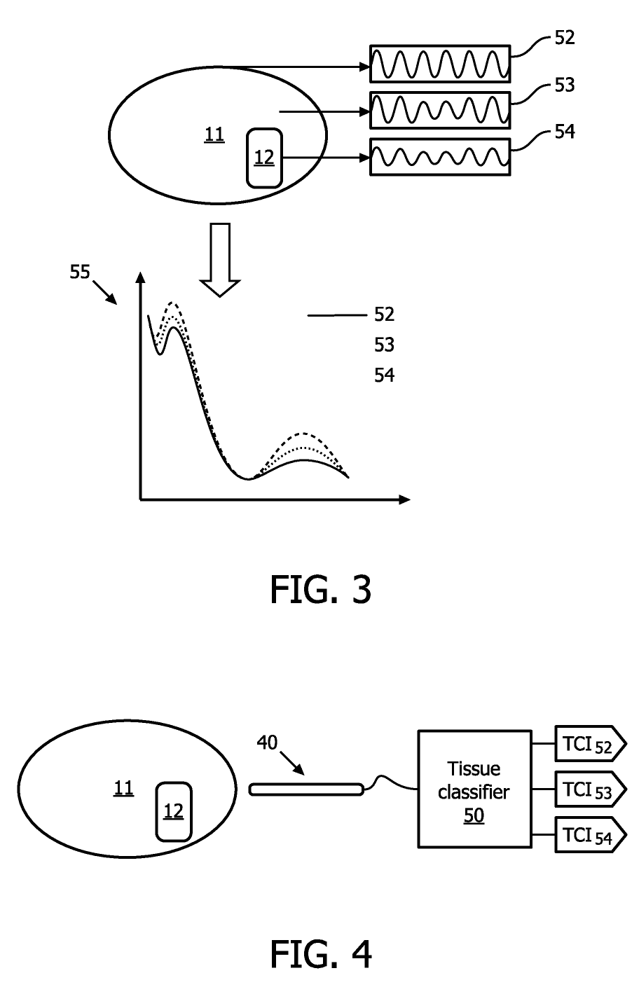

FIGS. 3 and 4 illustrate an exemplary embodiment of a tissue classification method in accordance with the present invention.

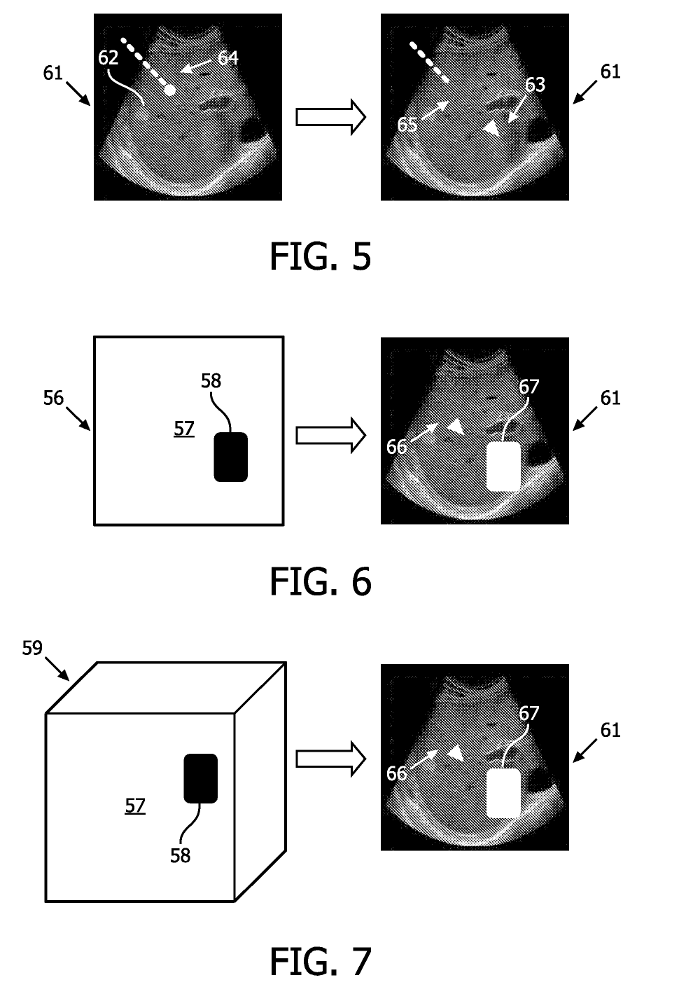

FIGS. 5-7 illustrate exemplary navigational guides in accordance with the present invention.

To facilitate an understanding of the present invention, exemplary embodiments of the present invention will be provided herein directed to a tool navigation system shown in FIG. 1.

Referring to FIG. 1, the tool navigation system employs an ultrasound probe 20, an ultrasound imager 21, an optional preoperative scanner 30, an interventional tool 40, a tool tracker 41 having one or more optional positions sensors 42, a tissue classifier 50 having one or more optional tissue sensors 51, and an image navigator 60.

Ultrasound probe 20 is any device as known in the art for scanning an anatomical region of a patient via acoustic energy (e.g., scanning an anatomical region 11 of a patient 10 as shown in FIG. 1). Examples of ultrasound probe 20 include, but are not limited to, a two-dimensional (“2D”) ultrasound probe having a one-dimensional (“1D”) transducer array.

Ultrasound imager 21 is a structural configuration of hardware, software, firmware and/or circuitry as known in the art for generating an ultrasound image of the anatomical region of the patient as scanned by ultrasound probe 20 (e.g., an ultrasound image 61 of a liver as shown in FIG. 1).

Preoperative scanner 30 is a structural configuration of hardware, software, firmware and/or circuitry as known in the art for generating a preoperative volume of the anatomical region of the patient as scanned by a preoperative imaging modality (e.g., magnetic resonance imaging, computed tomography imaging and x-ray imaging).

Interventional tool 40 is any tool as known in the art for performing minimally invasive procedures involving a navigation of interventional tool 40 within the anatomical region. Examples of interventional tool 40 include, but are not limited to, a needle and a catheter.

Tool tracker 41 is a structural configuration of hardware, software, firmware and/or circuitry as known in the art for tracking a position of interventional tool 40 relative to the ultrasound image of the anatomical region. To this end, interventional tool 40 can be equipped with position sensor(s) 42 as known in the art including, but are not limited to, acoustic sensors(s), ultrasound transducer(s), electromagnetic sensor(s), optical sensor(s) and/or optical fiber(s).

In one exemplary embodiment of tool tracker 41, a spatial position of a distal tip of interventional tool 40 with respect to a global frame of reference attached to the ultrasound image is the basis for position tracking interventional tool 40. Specifically, position sensor(s) 42 in the form of acoustic sensor(s) at a distal tip of interventional tool 40 receive(s) signal(s) from ultrasound probe 20 as ultrasound probe 20 beam sweep a field of view of the anatomical region. The acoustic sensor(s) provide acoustic sensing waveforms to tool tracker 41, which in turns executes a profile analysis of the acoustic sensing waveforms. Particularly, for the acoustic sensing waveforms, a time of arrival of the ultrasound beams indicate a distance of the acoustic sensor(s) to the imaging array, and an amplitude profile of the ultrasound beam indicate a lateral or an angular distance of the acoustic sensor(s) to an imaging array of the ultrasound probe.

Tissue classifier 50 is a structural configuration of hardware, software, firmware and/or circuitry as known in the art or as provided by the present invention for characterizing tissue within the ultrasound image of the anatomical region. For example, as shown in FIG. 1, tissue classifier 50 can characterize unhealthy tissue 63 within healthy tissue 62 as shown in an ultrasound image 61 of an anatomical region (e.g., a liver of the patient).

In practice, tissue classifier 50 can be operated in one or more various modes including, but not limited to, a tool signal mode utilizing tissue sensor(s) 51 and an image mode utilizing an imaging device (e.g., preoperative scanner 30).

Tool Signal Modes.

For this mode, tissue sensor(s) 42 are embedded in/attached to interventional tool 40, particularly at the tip of interventional tool 40, for sensing tissue adjacent interventional tool 40 as interventional tool 40 is navigated within the anatomical region to the target location. In practice, one or more sensors can serve as both a tissue sensor 42 and position sensor 51.

In one exemplary embodiment of a tool signal mode, tissue sensor(s) 42 is an ultrasound transducer as known in the art serving as an acoustic sensor of interventional tool 40 and for measuring acoustic characteristics of tissue adjacent a distal tip of interventional tool 40. For example, the ultrasound transducer can be utilized for pulse-echo signal analysis by tissue classifier 50 whereby an operating frequency of the ultrasound transducer is few millimeters of tissue encircling the distal tip of interventional tool 40 (e.g., in the 20 to 40 MHz range). Note that such a high frequency element is easily embedded into interventional tool 40, because of the small dimensions, and is still able to receive signals from the lower frequency (˜3 MHz) ultrasound probe 20 in the hydrostatic regime. Characteristics of the pulse-echo signal, for instance the frequency dependent attenuation as measured by temporal filtering and fitting of the detected envelope of the signal, are used by tissue classifier 50 for tissue classification. Two orthogonal or angled ultrasound transducers can be used to measure anisotropy of the medium (e.g. relevant to epidural injections, the ligament is highly anisotropic but the epidural space is isotropic).

In a second exemplary embodiment of the tool signal mode, tissue sensor(s) 42 is a PZT microsensor as known in the art for measuring acoustic impedance of the tissue adjacent the distal tip of interventional tool 40. For example, an acoustic impedance of a load in contact with the distal tip of interventional tool 40 changes as interventional tool 40 traverses different tissue types. The load changes results in a corresponding change in a magnitude and a frequency of a resonant peak of the PZT mircosensor, which is used by tissue classifier 50 for tissue classification.

In a third exemplary embodiment of the tool signal mode, tissue sensor(s) 42 is an fiber optic hydrophone as known in the art. For example, optical spectroscopy technique as known in the art involves an optical fiber delivering light to the tissue encircling the distal tip of interventional tool 40 and operating as a hydrophone to provide tissue differentiation information to tissue classifier 50.

In practice for any tool signal mode, tissue classifier 50 working on signal characteristics can first be trained on many anatomical regions with known tissue types and the best signal parameters are used in combination to output the probability to be in one of the following pre-determined tissue types including, but not limited to, skin, muscle, fat, blood, nerve and tumor. For example as shown in FIG. 3, the tissue sensing device at the distal tip of interventional tool 40 provides a signal 52 indicative of the tissue being skin of anatomical region 11, a signal 53 indicative of the signal being normal tissue of anatomical region 11, and a signal 54 indicative of tissue being a tumor 12 of anatomical region 11. Tissue classifier 50 is trained to identify a sharp change in a signal characteristic which is indicative of crossing of a tissue boundary. A training graph 55 is representative of identifiable changes in signals 52-54.

Image Modes.

For this mode, a spatial map of a tissue characterization of the anatomical region is generated by tissue classifier 50 dependent upon an imaging modality being utilized for this mode.

In a photo-acoustic exemplary embodiment, interactions between acoustic energy and certain wavelengths in light are exploited by tissue classifier 50 as known in the art to estimate tissue specific details of the anatomical region. Specifically, the mode involves an emission of acoustic energy and measurement of optical signatures of the resultant phenomenon, or vice versa. When integrated together the acoustic sensor(s) and the ultrasound image of the anatomical region, tissue classifier 50 generates spatial map of the tissue characterization that can be super-imposed to the ultrasound image of the anatomical region.

In an echo-based spectroscopy exemplary embodiment, tissue classifier 50 implements techniques that look at high resolution raw radio-frequency (“RF”) data to create B'mode ultrasound image of the anatomical region and their temporal variations can be utilized for adding additional tissue characterization details. Examples of a technique is elastography, which may detect certain types of cancerous legions based on temportal changes of the RF traces under micro-palpitations of the tissue. Other modes can be extensions of these techniques where they can use the temporal variations of the RF data to estimate tissue properties in the ultrasound image of the anatomical region.

In a preoperative tissue map mode, tissue classifier 50 generates a 2D or 3D pre-operative map of the tissue properties based on pre-operative image of the anatomical region provided by preoperative scanner 30 (e.g., MR spectroscopy). Alternately, tissue classifier 50 can obtain a tissue characterization map can be obtained from a large population studies on a group of pre-operative images of the anatomical region, which suggests any regions inside the tissue that have a higher likelihood of developing disease. Additionally, tissue classifier 50 can obtain a tissue characterization map from histo-pathology techniques as known in the art.

Still referring to FIG. 1, image navigator 60 is a structural configuration of hardware, software, firmware and/or circuitry as known in the art for displaying a navigational guide (not shown) relative to a display of ultrasound image 61 of the anatomical region. The navigational guide simultaneously illustrates a position tracking of interventional tool 40 by the tool tracker 41 and a tissue characterization of the anatomical region by tissue classifier 50. In practice, various display techniques as known in the art can be implemented for generating the navigation guide including, but not limited to, overlays, side-by-side, color coding, time series tablet and beamed to big monitor. In particular, the navigational guide can include graphical icons and/or tissue characterizations maps as will be further described in the context of FIG. 2.

Referring to FIG. 2, an operational method of the tool navigation shown in FIG. 1 will now be described herein. Upon initiation of the operational method, the operational method involves a continual execution of an anatomical imaging stage S70 of the anatomical region by ultrasound imager 21 as known in the art and of a tool tracking stage S71 of interventional tool 40 relative to the anatomical region by tool tracker 41 as known in the art.

A tissue classifying stage S72 is executed as needed to characterize tissue within the ultrasound image of the anatomical region. For example, as previously stated herein, tissue classifier 50 can characterize unhealthy tissue 63 within healthy tissue 62 as shown in an ultrasound image 61 of an anatomical region (e.g., a liver of the patient). More particularly for tissue classifying stage 72, tissue classifier 50 characterizes tissue within the ultrasound image of the anatomical region dependent upon the applicable tool signal mode(s) and/or image mode(s) of tissue classifier 50.

For the tool signal mode(s), as shown in FIG. 4, tissue classifier 50 can read the signal from interventional tool 40 to thereby communicate a tissue classification signal TCI indicative of the tissue being skin of anatomical region, normal tissue of anatomical region, or a tumor of anatomical region. During an image navigating stage S73 (FIG. 1), image navigator 60 processes tissue classification signal TCI to generate a graphical icon illustrating a position tracking of interventional tool 40 by the tool tracker 41 and a tissue characterization of the anatomical region by tissue classifier 50.

In practice, image navigator 60 modulates one or more(s) features of the graphical icon to indicate when interventional tool 40 as being tracked is adjacent tumorous tissue. For example, as shown in FIG. 5, a graphical icon 64 in the form of a rounded arrow can be overlain on ultrasound image 61 as the tracked position of interventional tool 40 indicates the distal tip of interventional tool is adjacent normal tissue, and a graphical icon 65 in the form of a pointed arrow can be overlain on ultrasound image 61 as the tracked position of interventional tool 40 indicates the distal tip of interventional tool 40 is adjacent tumorous tissue. Other additional modulations to a graphical icon may alternatively or concurrently be implemented including, but not limited to, color changes of the graphical icon or a substitution of a different graphical icon.

More particularly, a shape of head of the arrow indicates the type of tissue currently adjacent a distal tip of interventional tool 40 and a shaft of the arrow indicates a path of interventional tool 40 through the anatomical region. Additionally, the shaft of the arrow can be color coded to indicate the type of tissue along the path of interventional tool 40. Moreover, to facilitate multiple samplings of the anatomical region, markers (not shown) can be used to indicate a previously sampled location.

For image mode(s), tissue classifier 50 generates and communicates a spatial map of the tissue characterization of the anatomical region to image navigator 60, which in turn overlays the tissue characterization map on the ultrasound image. For example, FIG. 6, illustrates a 2D spatial map 56 of normal tissue 57 encircling tumorous tissue 58. In this example, the 2D spatial map was generated by tissue classifier 50 via a photo-acoustic mode and/or an echo-based spectroscopy. During image navigating stage S73, image navigator 60 overlays 2D spatial map on ultrasound image 61 with a graphical icon 66 indicative of the position tracking of interventional tool 40 and a graphical icon 67 indicative of the tumorous tissue 58.

Also by example, as shown in FIG. 7, tissue classifier 50 can derive 2D spatial map 56 (FIG. 6) from a registration of a 3D spatial map 59 of the tissue characterization of the anatomical region derived from a pre-operative image of the anatomical region generated by preoperative scanner 30.

Referring back to FIG. 1, in practice, ultrasound imager 21, optional preoperative scanner 30, tool tracker 41, tissue classifier 50 and image navigator 60 can be installed as known in the art on a single workstation or distributed across a plurality of workstations (e.g., a network of workstations).

Referring to FIGS. 1-7, those having ordinary skill in the art will appreciate in view of the teachings provided herein that numerous benefits of the present invention including, but not limited to, providing a clinician a rich source of information for facilitating better judgment of each patient, personalizing the treatment regimen, and keeping better control of where tissue samples are obtained from or control the region where a certain drug is injected.

While various exemplary embodiments of the present invention have been illustrated and described, it will be understood by one having ordinary skill in the art in view of the teachings provided herein that the exemplary embodiments of the present invention as described herein are illustrative, and various changes and modifications can be made and equivalents can be substituted for elements thereof without departing from the true scope of the present invention. In addition, many modifications can be made to adapt the teachings of the present invention without departing from its central scope. Therefore, it is intended that the present invention not be limited to the particular exemplary embodiments disclosed as the best mode contemplated for carrying out the present invention, but that the present invention includes all embodiments falling within the scope of the appended claims.

1. A tool navigation system, comprising:
an ultrasound probe operable to scan an anatomical region;
an ultrasound imager operably connected to the ultrasound probe to generate an ultrasound image of the anatomical region responsive to a scan of the anatomical region by the ultrasound probe;
an interventional tool operable to be navigated within the anatomical region;
a tool tracker operably connected to the interventional tool to track a position of the interventional tool relative to the anatomical region as the interventional tool is navigated within the anatomical region;
a tissue classifier operably connected to at least one of the ultrasound probe, the interventional tool and the tool tracker to characterize tissue of the anatomical region adjacent the interventional tool as the interventional tool is navigated within the anatomical region; and
an image navigator operably connected to the ultrasound imager, the tool tracker and the tissue classifier to display a navigational guide relative to a display of the ultrasound image of the anatomical region,
wherein the navigational guide illustrates a position tracking by the tool tracker of the interventional tool relative to the anatomical region for spatial guidance of the interventional tool within the anatomical region, and
wherein the navigational guide further illustrates a tissue characterization by the tissue classifier of the tissue of the anatomical region adjacent the interventional tool for target guidance of the interventional tool to a target location within the anatomical region.

2. The tool navigation system of claim 1, further comprising:
at least one position sensor operably connecting the tool tracker to the interventional tool to facilitate the position tracking by the tool tracker of the interventional tool relative to the anatomical region,
wherein the at least one position sensor is operable to sense at least one of acoustic energy, electromagnetic energy or optical energy indicative of the position of the interventional tool relative to the anatomical region.

3. The tool navigation system of claim 2,
wherein each position sensor comprises at least one ultrasound transducer operable to generate an acoustic sensing waveform indicative of an acoustic sensing of a scan of the anatomical region by ultrasound probe; and
wherein the tool tracker is operable to execute a profile analysis of the at least one acoustic sensing waveform as a basis for acoustically tracking the position of the interventional tool relative to the anatomical region as the interventional tool is navigated within the anatomical region.

4. The tool navigation system of claim 3, wherein the at least one position sensor includes at least one of a co-polymer ultrasound transducer, a piezoelectric sensor, a capacitive micro-machined ultrasonic transducer, or a fiber optic hydrophone.

5. The tool navigation system of claim 1, further comprising:
at least one tissue sensor operably connecting the tissue classifier to the interventional tool to facilitate a tissue characterization by the tissue classifier of the tissue of the anatomical region adjacent the interventional tool.

6. The tool navigation system of claim 5, wherein the at least one tissue sensor includes at least one of a fiber optic hydrophone, a piezoelectric sensor and a capacitive micro-machined ultrasonic transducer.

7. The tool navigation system of claim 5, wherein each tissue sensor operably connects the tool tracker to the interventional tool to facilitate the position tracking by the tool tracker of the interventional tool relative to the anatomical region.

8. The tool navigation system of claim 1,
wherein the navigation guide comprises a graphical icon of the interventional tool illustrating at least one of the position tracking of the interventional tool by the tool tracker or the tissue characterization of the anatomical region by the tissue classifier; and
wherein the image navigator is operable to modulate at least one feature of the graphical icon responsive to any change to a tissue type of the tissue characterization of the anatomical region by the tissue classifier.

9. The tool navigation system of claim 8, wherein the graphical icon comprises an arrow having an least one feature dependent upon any change to a tissue type of the tissue characterization of the anatomical region by the tissue classifier.

10. The tool navigation system of claim 9,
wherein a shaft of the arrow illustrates position tracking of the interventional tool by the tool tracker, and
wherein at least one of a head of the arrow or the shaft of the arrow illustrate the tissue characterization of the anatomical region by the tissue classifier.

11. The tool navigation system of claim 1, wherein the navigation guide includes at least one graphical icon illustrating a sampled location of the anatomical region.

12. The tool navigation system of claim 1,
wherein the tissue classifier is operably connected to at least one of the ultrasound imager to generate a spatial tissue characterization map of the anatomical region including a plurality of tissue types of the anatomical region; and
wherein the navigation guide includes
the spatial tissue characterization map, and
a graphical icon of the interventional tool illustrating the position tracking of the interventional tool by the tool tracker.

13. The tool navigation system of claim 1, further comprising:
a pre-operative scanner operable to generate a pre-operative image of the anatomical region,
wherein the tissue classifier is operably connected to the pre-operative scanner to generate a spatial tissue characterization map of the anatomical region from the pre-operative image of the anatomical region,
wherein the spatial tissue characterization map of the anatomical region includes a plurality of tissue types of the anatomical region; and
wherein the navigation guide includes
the spatial tissue characterization map, and
a graphical icon of the interventional tool illustrating the position tracking of the interventional tool by the tool tracker.

14. A tool navigation system, comprising:
an ultrasound imager operably connected to an ultrasound probe to generate an ultrasound image of an anatomical region responsive to a scan of the anatomical region by the ultrasound probe;
a tool tracker, operably connected to an interventional tool operable to be navigated within the anatomical region, to track a position of the interventional tool relative to the anatomical region as the interventional tool is navigated within the anatomical region;
a tissue classifier operably connected to at least one of the ultrasound probe, the interventional tool or the tool tracker to characterize tissue of the anatomical region adjacent the interventional tool as the interventional tool is navigated within the anatomical region; and
an image navigator operably connected to the ultrasound imager, the tool tracker and the tissue classifier to display a navigational guide relative to a display of the ultrasound image of the anatomical region,
wherein the navigational guide illustrates a position tracking by the tool tracker of the interventional tool relative to the anatomical region for spatial guidance of the interventional tool within the anatomical region, and
wherein the navigational guide further illustrates a tissue characterization by the tissue classifier of the tissue of the anatomical region adjacent the interventional tool for target guidance of the interventional tool to a target location within the anatomical region.

15. The tool navigation system of claim 14, further comprising:
at least one position sensor operably connecting the tool tracker to the interventional tool to facilitate the position tracking by the tool tracker of the interventional tool relative to the anatomical region,
wherein the at least one position sensor is operable to sense at least one of acoustic energy, electromagnetic energy or optical energy indicative of the position of the interventional tool relative to the anatomical region,
wherein each position sensor comprises at least one ultrasound transducer operable to generate an acoustic sensing waveform indicative of an acoustic sensing of a scan of the anatomical region by ultrasound probe, and
wherein the tool tracker is operable to execute a profile analysis of the at least one acoustic sensing waveform as a basis for acoustically tracking the position of the interventional tool relative to the anatomical region as the interventional tool is navigated within the anatomical region.

16. The tool navigation system of claim 14,
wherein the navigation guide comprises a graphical icon of the interventional tool illustrating at least one of the position tracking of the interventional tool by the tool tracker or the tissue characterization of the anatomical region by the tissue classifier; and
wherein the image navigator is operable to modulate at least one feature of the graphical icon responsive to any change to a tissue type of the tissue characterization of the anatomical region by the tissue classifier.

17. The tool navigation system of claim 16, wherein the graphical icon comprises an arrow having an least one feature dependent upon any change to a tissue type of the tissue characterization of the anatomical region by the tissue classifier.

18. A tool navigation method, comprising:
generating an ultrasound image of an anatomical region from a scan of the anatomical region by an ultrasound probe;
tracking a position of an interventional tool relative to the anatomical region as the interventional tool is navigated within the anatomical region;
characterizing tissue of the anatomical region adjacent the interventional tool as the interventional tool is navigated within the anatomical region; and
displaying a navigational guide relative to a display of the ultrasound image of the anatomical region,
wherein the navigational guide illustrates the position tracking of the interventional tool relative to the anatomical region for spatial guidance of the interventional tool within the anatomical region, and
wherein the navigational guide further illustrates the tissue characterization of the anatomical region for target guidance of the interventional tool to a target location within the anatomical region.

19. The tool navigation method of claim 18, wherein the navigation guide includes at least one of (i) a spatial tissue characterization map of the anatomical region, or (ii) a graphical icon of the interventional tool illustrating at least one of the position tracking of the interventional tool by the tool tracker or the tissue characterization of the anatomical region by the tissue classifier.

20. The tool navigation method of claim 18, further comprising:
modulating at least one feature of a graphical icon responsive to any change to a tissue type of the tissue characterization of the anatomical region.
